(19) United States
(12) Patent Application Publication
(10) Pub. No.: US 2026/0081131 A1
(43) Pub. Date: Mar. 19, 2026

(54) SYSTEM AND METHOD FOR DETECTING WAFERS OUTGASSING IN A VACUUM LOAD-LOCK USING A RESIDUAL GAS SENSOR

(71) Applicant: Applied Materials Israel Ltd., Rehovot (IL)

(72) Inventors: Irit Ruach Nir, Rehovot (IL); Sven Ruhle, Tel Aviv (IL)

(73) Assignee: Applied Materials Israel Ltd., Rehovot (IL)

(21) Appl. No.: 18/890,643

(22) Filed: Sep. 19, 2024

(51) Int. Cl. H01J 49/40; G01N 33/00

(52) U.S. Cl. CPC H01J 49/40; G01N 33/0063

(57) ABSTRACT

A system for detecting and/or minimizing outgassing contamination released from a sample into a tool, the system including: a tool configured to characterize a tested sample, wherein operation conditions of the characterization tool includes reaching a vacuum level of 10⁻⁶ Torr or higher, a load lock chamber, and a time-of-flight residual gas analyzer (TOF-RGA) sensor in fluid flow communication with the load-lock chamber, the TOF-RGA is configured to provide a real-time measurement of a composition of gasses released from the tested sample into the load lock chamber.

### TECHNICAL FIELD

The present disclosure relates generally to manufacturing and metrology of specimens.

### BACKGROUND OF THE INVENTION

Specimens, such as but not limited to, wafers and semiconductors, are typically manufactured and inspected within vacuum environments, such as those found in semiconductor device production processes. Tools operating under vacuum conditions, including scanning electron microscopes (SEMs), are commonly utilized for specimen characterization, wherein each specimen is accommodated in a load-lock chamber and transferred into a vacuum chamber of the tool. The insertion of specimens into these tools often requires gas pumping to achieve the required vacuum levels. However, such samples often undergo outgassing emission of gases therefrom, e.g., including during the pumping process. The outgassing from the samples may result in tool contamination, cross-contamination between samples, diminished tool health and performance, and reduced high-voltage immunity.

Hence, there is a need in the art for methods for rapid detection of outgassing from samples prior to insertion into the high vacuum environment, i.e. in the load lock.

### BRIEF SUMMARY OF THE INVENTION

Aspects of the disclosure, according to some embodiments thereof, relate to manufacturing and metrology of specimens.

More specifically, but not exclusively, aspects of the disclosure, according to some embodiments thereof, relate to manufacturing and characterization of specimens, such as wafers, photoresists, semiconductor devices and/or components thereof.

Thus, according to an aspect of some embodiments, there is provided a system for detecting/minimizing outgassing contamination released from a tested sample into a characterization tool.

Advantageously, according to some embodiments, the disclosed system facilitates minimizing the outgassing contamination emitted into a process chamber of the tool, thereby minimizing cross-contamination, and facilitating health and performance of the tool.

Advantageously, according to some embodiments, the disclosed system enables rapid outgassing detection.

Advantageously, according to some embodiments, the disclosed system enables distinguishing between water and organic outgassing.

According to some embodiments, there is provided a system for detecting and/or minimizing outgassing contamination released from a sample into a characterization tool, the system including:
a main chamber including a characterization tool configured to characterize a sample, wherein operation conditions of the characterization tool include reaching a vacuum level of 10⁻⁵ Torr or higher;
a load-lock chamber configured to receive the sample;
a time-of-flight residual gas analyzer (TOF-RGA) sensor in fluid flow communication with the load-lock chamber, wherein the TOF-RGA sensor is configured to provide a real-time measurement of an amount of outgassing released from the tested sample in the load-lock chamber; and
and a controller configured to receive measurements from the TOF-RGA sensor, to compute an amount of outgassing detected in the load-lock chamber and to trigger transfer of the sample from the load-lock chamber to the main chamber, only if the amount of outgassing detected in the load-lock chamber is at or below a predetermined threshold, thereby reducing a risk for cross-contamination of other/next tested samples and facilitating maintaining tool health, and to transfer the sample into the characterization chamber.

According to some embodiments, computing the amount of outgassing includes computing a sum of a predetermined range of peaks. According to some embodiments, the computing includes distinguishing between water and organic outgassing,

According to some embodiments, the system further includes a pump configured to generate a vacuum in the load-lock chamber, and a pressure monitor configured to monitor the pressure within the load-lock chamber. According to some embodiments, the controller is configured to trigger measurements by the TOF-RGA sensor, when signals provided by the pressure monitor indicate that a first predetermined pressure level has been obtained. According to some embodiments, the controller is configured to trigger transfer of the sample when signals provided by the pressure monitor indicate that a second predetermined pressure, essentially equal to the pressure in the main chamber, or when signals provided by the pressure monitor indicate that a predetermined third threshold value has been reached; or when signals provided by the pressure monitor indicate that a predetermined amount of time has passed.

According to some embodiments, the outgassing sensitivity of the TOF-RGA sensor is in a range of 10⁻⁴ Torr to 10⁻¹⁰ Torr.

According to some embodiments, the measurement time of the TOF-RGA sensor to provide the composition of gases is 30 seconds or less. According to some embodiments, the measurement time of the TOF-RGA sensor to provide the composition of gases is 5 seconds or less.

According to some embodiments, the TOF-RGA sensor provides the real-time measurement of the composition of gases having a pressure of up to 10⁻⁴ Torr.

According to some embodiments, the TOF-RGA sensor is replaceable.

According to some embodiments, the characterization tool is selected from: a scanning electron microscope (SEM), a focused ion beam (FIB), a transmission electron microscope (TEM), a scanning probe microscope (SPM). Each possibility is a separate embodiment.

According to some embodiments, the tested sample includes a photoresist or an organic residue from a previous process step.

According to some embodiments, the controller is further configured to trigger an alert if the amount of outgassing exceeds the predetermined threshold for a predetermined amount of time.

According to some embodiments, there is provided a method for detecting and/or minimizing outgassing from a sample, the method including:
positioning a tested sample into a load-lock chamber of a characterization tool;
activating a pump associated with the load-lock chamber to achieve a first pre-determined vacuum level in the load-lock;
upon reaching the first pre-determined vacuum level, performing a residual mass analysis by a TOF-RGA sensor fluidly connected to the load-lock chamber, to identify an amount of gasses released from the tested sample into the load-lock chamber; and
transferring the sample into a main chamber of the characterization tool only if the amount of gasses detected is below a predetermined threshold value, thereby reducing a risk for cross-contamination of other/next tested samples and facilitating maintaining tool health.

According to some embodiments, the method further includes maintaining the tested sample in the load-lock chamber for a predetermined amount of time, if the amount of gasses detected is above the predetermined threshold value.

According to some embodiments, transferring the sample into the main chamber of the characterization tool, if during the predetermined amount of time, the amount of gasses detected is at or below the predetermined threshold value.

According to some embodiments, removing the sample from the load-lock chamber, if during the predetermined amount of time, the amount of gasses detected remains above the predetermined threshold value.

According to some embodiments, computing the amount of outgassing includes computing a sum of a predetermined range of peaks.

According to some embodiments, the sample is a wafer.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1 presents a block diagram of a system for detecting and/or minimizing outgassing contamination released into a tool, according to some embodiments;

FIGS. 2A and 2B present a flowchart of a method for inserting a specimen into a vacuum system through a load lock, according to some embodiments;

FIG. 3 is an example of an experimentally obtained plot of a partial pressure as a function of mass/z in units of atomic mass (amu) detected by a TOF-RGA sensor positioned on, and being in fluid flow communication with a vacuum chamber, according to some embodiments; and

FIG. 4 is an example of an experimentally obtained plot of a partial pressure as a function of mass/z in units of atomic mass (amu) detected by a quadrupole-RGA sensor positioned on, and being in fluid flow communication with a vacuum, according to some embodiments.

### DETAILED DESCRIPTION OF THE INVENTION

The principles, uses, and implementations of the teachings herein may be better understood with reference to the accompanying description and figures. Upon perusal of the description and figures present herein, one skilled in the art will be able to implement the teachings herein without undue effort or experimentation.

According to an aspect of some embodiments, there is provided a system for detecting, controlling and/or minimizing organic outgassing contamination released from a specimen into a tool, such as a characterization/metrology tool, a manufacturing tool, and the like.

According to an aspect of some embodiments, there is provided a method for detecting, controlling and/or minimizing organic outgassing contamination released into a tool, such as a characterization/metrology tool, a manufacturing tool, and the like.

According to an aspect of some embodiments, there is provided a method for manufacturing a semiconductor device, wherein the method includes detecting, controlling and/or minimizing organic outgassing contamination released into a tool.

Advantageously, in some embodiments, the disclosed system and method are configured to provide a rapid analysis of outgassing released/emitted from a sample.

As used herein, according to some embodiments, the terms “sample” and “specimen” may be interchangeable.

As used herein, according to some embodiments, the term “specimen” may refer to any sample suitable for undergoing a process or an inspection in a tool operation under vacuum environment. According to some embodiments, the term “specimen” may refer to a semiconductor device and/or a component/element thereof. According to some embodiments, the term “specimen” may refer to a wafer (e.g., a Si wafer, a GaAs wafer, and the like), a diode, a transistor, an integrated circuit, and the like, and/or any component/element thereof. According to some embodiments, the term “specimen” may refer to an electronic device and/or components/elements thereof. According to some embodiments, the term “specimen” may refer to an energy store device or a component/element thereof. According to some embodiments, the term “specimen” may refer to an optoelectronic device or any component/element thereof. According to some embodiments, the term “specimen” may refer to a photoresist.

In some embodiments, results of the outgassing analysis may be obtained by about 5 seconds or less. Thereby, in some embodiments, the disclosed system and method facilitate a manufacturing process of a sample, while maintaining the performance and the health of the manufacturing/metrology tool. In some embodiments, the disclosed system and method may be substantially devoid of disruptions or impediments that delay the manufacturing/metrology process.

Advantageously, in some embodiments, the disclosed system and method are configured to distinguish between water and organic outgassing.

Advantageously, in some embodiments, the disclosed system and method have high sensitivity of outgassing detection. In some embodiments, the disclosed system and method are capable of detecting outgassing reaching about 10⁻⁵ to 10⁻¹⁰ Torr.

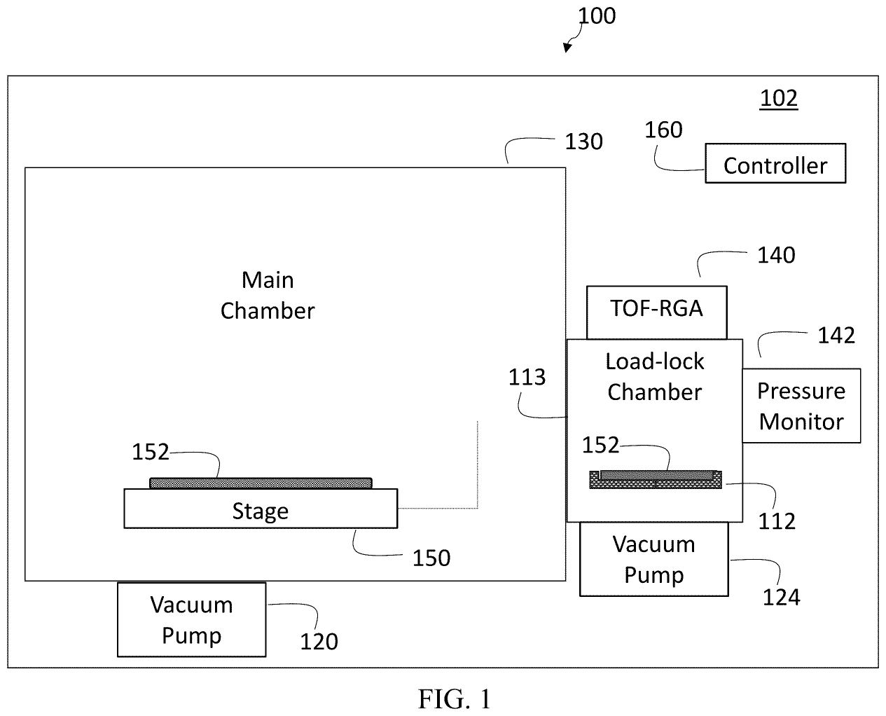

Reference is made to FIG. 1, which presents a block diagram of a system 100 for detecting, controlling and/or minimizing outgassing contamination released from a specimen into a tool, according to some embodiments. In some embodiments, system 100 may be utilized in characterizing and/or manufacturing a specimen.

System 100 includes a characterization tool 102. According to some embodiments, characterization tool 102 may include any type of a characterization/metrology tool and/or a process tool having operating conditions of reaching a vacuum level of about 10⁻⁶ Torr or higher, about 10⁻⁷ Torr or higher, about 10⁻⁸ Torr or higher, and about 10⁻⁹ Torr or higher. Each possibility is a separate embodiment.

According to some embodiments, characterization tool 102 may include any type of a characterization/metrology tool and/or process tool having a vacuum chamber and a load-lock (i.e., a load-lock chamber). According to some embodiments, characterization tool 102 may include any tool type including a chamber operating under or capable of reaching operating conditions in high vacuum (HV) and ultra-high vacuum (UHV) regime.

In some embodiments, characterization tool 102 may include a tool utilized in a process for manufacturing a specimen. According to some embodiments, characterization tool 102 may include a process tool configured to manufacture a semiconductor device or a component thereof. Alternatively, or additionally, in some embodiments, characterization tool 102 may be configured to characterize the specimen.

According to some embodiments, characterization tool 102 may include, among others, a scanning electron microscope (SEM), a high-resolution SEM (HR-SEM), a focused ion beam (FIB) setup, a transmission electron microscope (TEM), an X-ray photoelectron spectroscopy tool (XPS), an ultraviolet photoelectron spectroscopy (UPS) tool, any type of a scanning probe microscope (SPM), and the like. Each possibility is a separate embodiment. As a non-limiting example, characterization tool 102 may include a scanning tunneling microscope (STM). Each possibility is a separate embodiment.

According to some embodiments, characterization tool 102 may be configured to conduct one or more manufacturing processes on the specimen. According to some embodiments, characterization tool 102 may be configured to deposit one or more elements onto the specimen.

According to some embodiments, characterization tool 102 includes a main chamber 130. According to some embodiments, main chamber 130 may include, among others, a chemical vapor deposition (CVD) chamber, a physical vapor deposition (PVD) chamber, a plasma deposition chamber, an atomic layer deposition (ALD) chamber, a sputtering chamber, a thermal evaporation chamber, and the like. Each possibility is a separate embodiment. According to some embodiments, the processing tool and the characterization tool are separate tools.

According to some embodiments, main chamber 130 may include a characterization chamber. As a non-limiting example, main chamber 130 may include a vacuum chamber of a SEM, e.g., in which a tested specimen is being characterized/inspected.

In some embodiments, characterization tool 102 may optionally include or be in communication with an electron beam (e-beam) source. In some embodiments, main chamber 130 may optionally include one or more electron sensors/detectors. In some embodiments, main chamber 130 may include or be in communication with one or more additional sensors/detectors, such as, but not limited to, optical sensors, electrical sensors, and the like, or a combination thereof.

In some embodiments, characterization tool 102 may include a plurality of main chambers 130 (not depicted). In some embodiments, plurality of main chambers 130 may include one or more characterization chambers and/or one or more process chambers, or any combination thereof. Each possibility is a separate embodiment. As a non-limiting example, plurality of main chambers 130 may include a characterization chamber and a plurality of process chambers.

In some embodiments, main chamber 130 of characterization tool 102 may be maintained under a vacuum level of about 10⁻⁵ Torr or higher, about 10⁻⁶ Torr or higher, about 10⁻⁷ Torr or higher, about 10⁻⁸ Torr or higher, and about 10⁻⁹ Torr or higher. Each possibility is a separate embodiment. In some embodiments, main chamber 130 of characterization tool 102 may be maintained under an UHV regime. In some embodiments, main chamber 130 of characterization tool 102 may be maintained under a vacuum level of in a range of about 10⁻⁵ Torr to about 10⁻¹⁰.

According to some embodiments, and as schematically depicted in FIG. 1, main chamber 130 includes a stage 150 configured to accommodate a specimen 152. In some embodiments, stage 150 may be movable.

In some embodiments, specimen 152 is configured to undergo at least one process and/or analysis in characterization tool 102, such as but not limited to, a characterization/inspection process, manufacturing process (such as, but not limited to, a deposition process, an etching process, and the like), or a combination thereof.

In some embodiments, characterization tool 102 includes a vacuum pump 120 configured to reduce pressure within characterization tool 102. According to some embodiments, further includes a load-lock chamber 110 configured to receive specimen 152 prior to specimen 152 reaching main chamber 130. According to some embodiments, load-lock chamber 110 may be connected to main chamber 130 via a sealed door 113. According to some embodiments, load-lock chamber 110 includes a specimen holder 112 configured to hold specimen 152. Load-lock chamber 110 is associated with a second vacuum pump 124 configured to reduce vacuum in load-lock chamber 110.

Once specimen 152 is loaded into load-lock chamber 110, a pressure therein may be reduced by vacuum pump 124 to a first predefined value.

According to some embodiments, characterization tool 102 includes a time-of-flight residual gas analyzer (TOF-RGA) sensor 140. According to some embodiments, TOF-RGA sensor 140 may be positioned at any position/location on load-lock chamber 110. According to some embodiments, TOF-RGA sensor 140 is in constant gas flow connection with load-lock chamber 110. According to some embodiments, upon reaching the first predetermined pressure level, measurements by the TOF-RGA sensor 140 may be initiated, e.g. via commands received from a controller 160. According to some embodiments, the first predetermined pressure value is in a range of about 10⁻³ Torr to about 10⁻⁴ Torr. In some embodiments, the pressure within load-lock chamber 110 may be further reduced to a substantially equalize the pressure maintained within main chamber 130 (e.g. between about 10⁻⁵ Torr to about 10⁻⁸ Torr), optionally while TOF-RGA sensor 140 continues measuring.

According to some embodiments, TOF-RGA sensor 140 is configured to provide a real-time measurement of a composition of gases released from specimen 152. According to some embodiments, the measurement time of TOF-RGA sensor 140 to provide the composition of gases released from a specimen may be in a range of about 1 seconds to about 5 seconds, about 1 seconds to about 10 seconds, about 1 seconds to about 20 seconds, and about 1 seconds to about 30 seconds. Each possibility is a separate embodiment.

According to some embodiments, the measurement time of TOF-RGA sensor 140 to provide the composition of gases released from a specimen may be about 30 seconds or less, about 20 seconds or less, about 10 seconds or less, about 5 seconds or less, about 4 seconds or less, about 3 seconds or less, about 2 seconds or less, about 1 second or less. Each possibility is a separate embodiment.

In some embodiments, a sampling rate of TOF-RGA sensor 140 for acquiring a substantially complete mass spectrum of the gasses released from specimen 152 into load-lock 110 may be up to about 250 msec. According to some embodiments, utilizing system 100 in a production line, such as in a manufacturing process of a semiconductor device, may substantially be devoid of throughput time disruptions/impediments.

According to some embodiments, TOF-RGA sensor 140 is configured to distinguish between water/aqueous outgassing and organic outgassing. Thereby, in some embodiments, reducing a risk for cross-contamination of other/next specimen and facilitating maintaining health and performance of characterization tool 102.

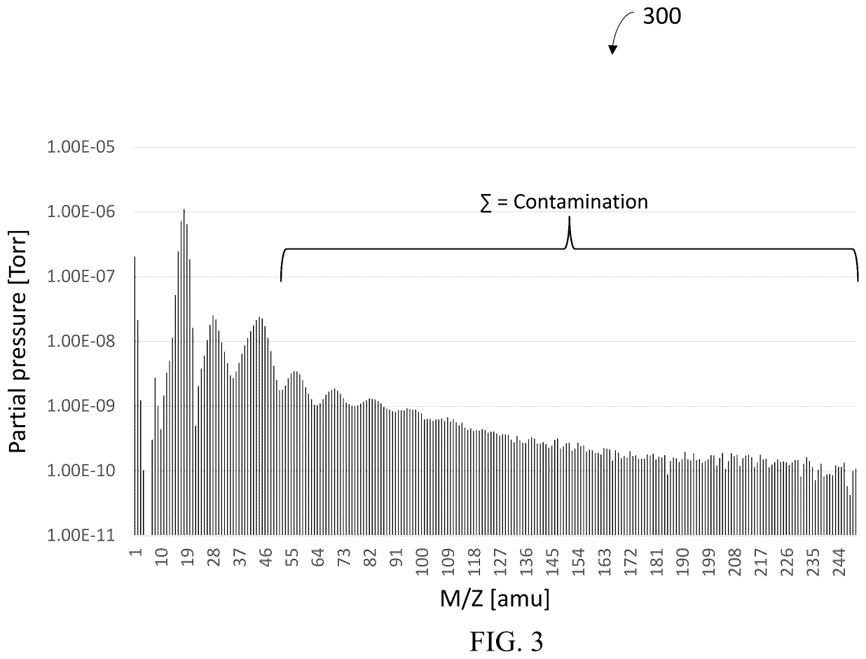

In some embodiments, TOF-RGA sensor 140 may be configured to monitor a peak of a specific gas molecule. Alternatively, or additionally, in some embodiments, TOF-RGA sensor 140 may be configured to monitor a range and/or a sum of peak ranges above a predefined value, e.g., as depicted in FIG. 3. As a non-limiting example, TOF-RGA sensor 140 may have a mass range of about 1-300 amu, and may be configured to summarize substantially all of predefined contaminations in a predefined range of the mass range thereof detected in the load-lock chamber.

In some embodiments, monitoring and/or detecting the sum of the range of peaks may advantageously reduce the detection time of the outgassing by TOF-RGA sensor 140. In some embodiments, the on/off switching of TOF-RGA sensor 140 may be rapid, e.g., in a range of about 2sec-5 minutes, between 1 sec to 1 minute, or between 1 sec and 30 seconds. Each possibility is a separate embodiment. Thereby, in some embodiments, facilitating swift vent to pump recovery and, in turn, optimizing turn-around time for outgassing detection between samples. In some embodiments, TOF-RGA sensor 140 may provide a high sampling rate of up to about 250 ms for obtaining a substantially complete outgassing mass spectrum.

In some embodiments, on/off switching (i.e., vent to pump recovery times between samples) of TOF-RGA sensor 140 may be 7sec to 5 min.

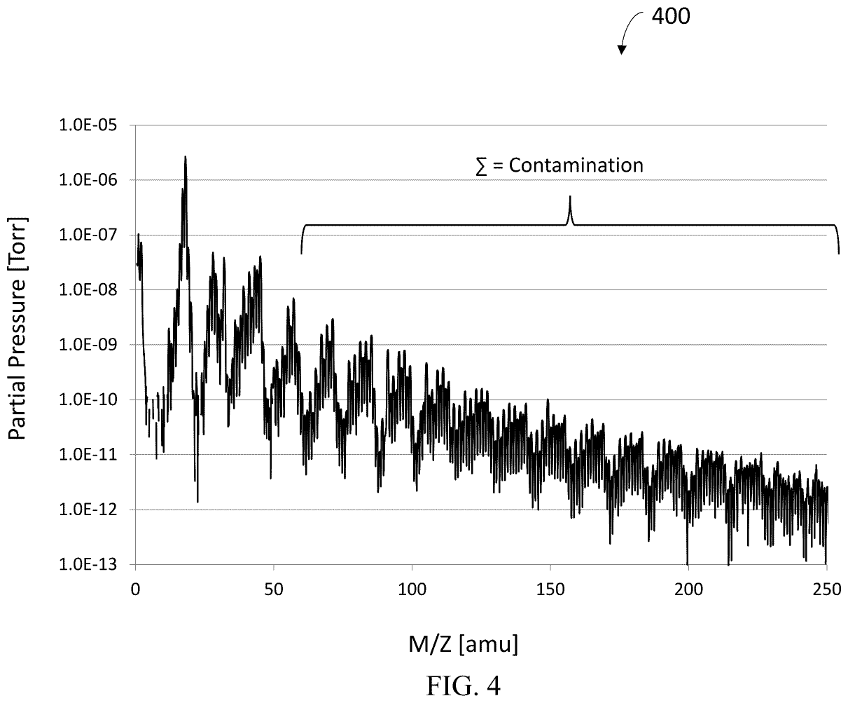

According to some embodiments, the residual mass analysis may include monitoring a peak of one or more of a specific gas composition (e.g., a specific gas molecule) having a predefined value, e.g., as depicted in FIG. 4. Thereby, in some embodiments, distinguishing between materials released/emitted from the specimen. Alternatively, or additionally, in some embodiments, the residual mass analysis may include monitoring a sum of range of peaks above a predefined value. Each possibility is a separate embodiment.

According to some embodiments, TOF-RGA sensor 140 provides the real-time measurement of the composition of gases having a pressure of up to about 10⁻⁴ Torr.

According to some embodiments, an outgassing detection capability/sensitivity of TOF-RGA sensor 140 is in a range of about 10⁻⁴ Torr to about 10⁻¹⁰ Torr. According to some embodiments, the outgassing capability/sensitivity of TOF-RGA sensor 140 is in a range of about 10⁻⁴ Torr to about 10⁻¹⁰ Torr, about 10⁻⁵ Torr to about 10⁻¹⁰ Torr, about 10⁻⁵ Torr to about 10-7 Torr, about 10⁻⁵ Torr to about 10⁻⁸ Torr, about 10⁻⁶ Torr to about 10⁻¹⁰ Torr, about 10⁻⁷ Torr to about 10⁻¹⁰ Torr, about 10⁻⁸ Torr to about 10⁻¹⁰ Torr, about 10⁻⁹ Torr to about 10⁻¹0 Torr. Each possibility is a separate embodiment.

In some embodiments, TOF-RGA sensor 140 provides gas analysis at a pressure range of about, about 10⁻³ Torr to about 10−4 Torr. Each possibility is a separate embodiment.

In some embodiments, TOF-RGA sensor 140 may be replaceable.

Advantageously, in some embodiments, TOF-RGA sensor 140 enables detecting outgassing from specimen 152 in load-lock chamber 110 prior transpiring specimen 152 into main chamber 130, thereby enabling opening door 113 between load-lock chamber 110 and main chamber 130 only once the detected outgassing is reduced to at or below a predefined pressure. Consequently, in some embodiments, outgassing in main chamber 130 is minimized and/or avoided thus reducing/minimizing a risk for reduced high-voltage immunity, and increasing health and/or performance of the tool.

According to some embodiments, characterization chamber 102 may include a pressure monitor 142 configured to monitor pressure (i.e., a vacuum level). In some embodiments, pressure monitor 142 may be configured to monitor the pressure in load-lock chamber 110.

According to some embodiments, controller 160 is configured to control the operation of system 100. More specifically, in some embodiments, controller 160 may be configured to control and/or synchronize the operation and/or functions of one or more of: load-lock chamber 110, main chamber 130, opening/closing of door 113, vacuum pump 120, TOF-RGA sensor 140 and/or stage 150.

According to some embodiments, controller 160 may be further configured to alert, e.g., a user, a different system or a component, when a sum of ranges of peaks above a predefined value is detected.

According to some embodiments, controller 160 may output instructions, e.g., to a user, a different system or a component. According to some embodiments, the instructions may include, among others, transferring the sample into the main chamber of the tool. According to some embodiments, the instructions may include, among others, removing the sample from the load-lock chamber, without transferring the sample into the main chamber of the tool. According to some embodiments, the instructions may include, among others, a time-out alert, as elaborated elsewhere herein.

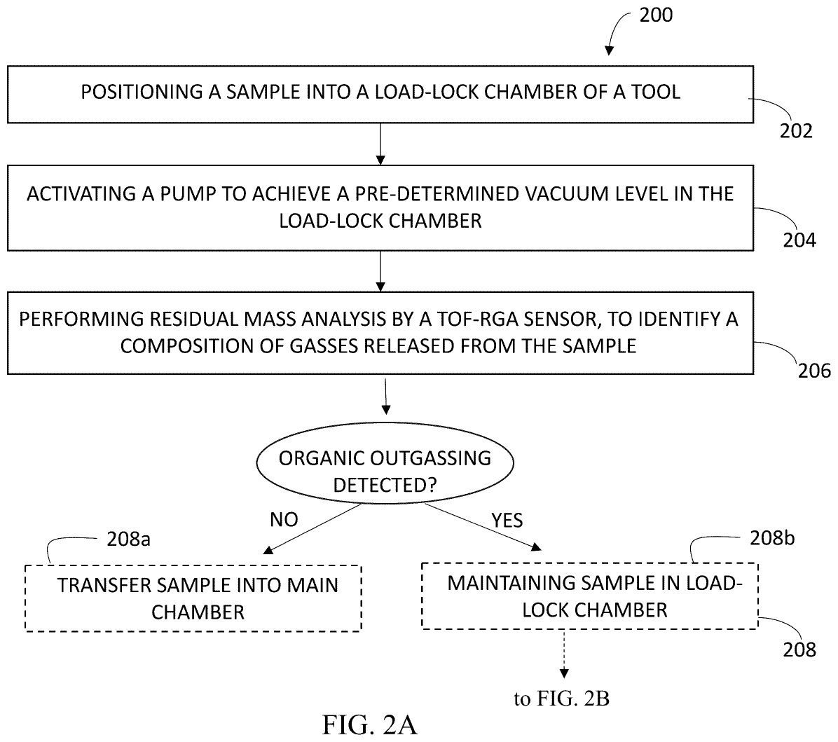

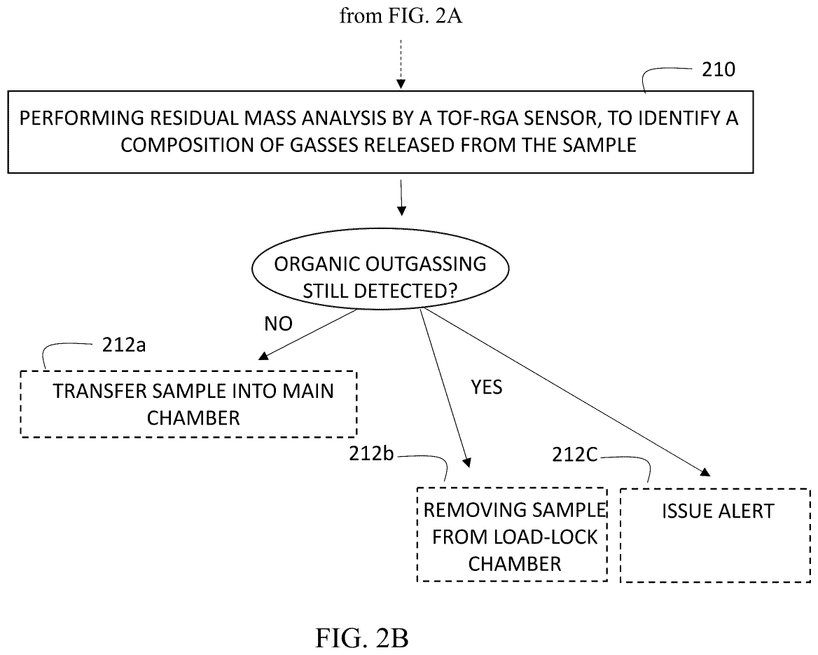

According to an aspect of some embodiments, there is provided a method for detecting outgassing from a sample. Reference is made to FIGS. 2A and 2B, which show a flowchart 200 of a method for detecting and/or minimizing outgassing, according to some embodiments.

According to some embodiments, the method may include a process for manufacturing a semiconductor device, in which an outgassing is being detected. According to some embodiments, the method may include characterizing a sample, such as but not limited to, a semiconductor device. Each possibility is a separate embodiment.

According to some embodiments, at step 202, the method includes positioning/loading a sample into a load-lock chamber of a characterization tool. In some embodiments, the characterization tool may include, among others, a SEM, a FIB, a TEM, an XPS, an UPS, a SPM (e.g., a STM), and the like. Each possibility is a separate embodiment. Alternatively, or additionally, in some embodiments, the characterization tool may include a process/manufacturing tool. According to some embodiments, the process/manufacturing tool may include, among others, a deposition/amber/tool, such as but not limited to, a CVD chamber/tool, a PVD chamber/tool, a plasma deposition chamber/tool, an ALD chamber/tool, a sputtering chamber/tool, a thermal evaporation chamber/tool, an etching chamber/tool, a cleaning chamber/tool, and the like, or any combination thereof. Each possibility is a separate embodiment. As a non-limiting example, the process/manufacturing tool may include a wafer manufacturing tool. As another non-limiting example, the process/manufacturing tool may include a semiconductor device manufacturing tool/chamber.

According to some embodiments, at step 204, the method includes periodically activating pumping and venting to achieve a pre-determined vacuum level in the load-lock chamber. According to some embodiments, the pre-determined vacuum level may be substantially equal to a vacuum level maintained in a main chamber of the tool, to facilitate operation of the main chamber and/or the tool. Alternatively, in some embodiments, the pre-determined vacuum level may be lower than the vacuum level maintained in the main chamber of the tool.

According to some embodiments, the pumping and venting may be performed by a vacuum pump of the tool.

According to some embodiments, step 204 may optionally include monitoring, by a pressure monitor of the tool, the pressure/vacuum level in the load-lock chamber. In some embodiments, the pressure monitor may conduct one or more measurements while the sample is being positioned within the load-lock chamber.

According to some embodiments, the sample may spontaneously undergo outgassing while being positioned within the load-lock chamber of the tool. According to some embodiments, the sample may undergo outgassing during the pumping and venting process.

According to some embodiments, at step 206, the method includes performing a residual mass analysis by TOF-RGA sensor, to identify, in real-time, a composition of gases released/emitted from the sample while being maintained within the load-lock chamber of the tool.

According to some embodiments, the residual mass analysis may include monitoring a peak of one or more of a specific gas composition (e.g., a specific gas molecule) having a predefined value. Alternatively, or additionally, in some embodiments, the residual mass analysis may include monitoring a sum of range of peaks above a predefined value. Each possibility is a separate embodiment.

In some embodiments, monitoring and/or detecting the sum of the range of peaks may advantageously reduce the detection time of the outgassing by the TOF-RGA sensor. In some embodiments, the on/off switching and subsequent stabilization/equilibration of the TOF-RGA sensor may be rapid, e.g., in a range of about 2 sec-5 minutes, typically in a range of 2 sec to 1 minute or in a range of 2 sec to 30 sec. Each possibility is a separate embodiment.

According to some embodiments, step 206 may further include distinguishing, in real-time, between water and organic outgassing released from the sample into the load-lock chamber by the TOF-RGA sensor. In some embodiments, detecting the composition of the gases released/emitted from the sample may include detecting a partial pressure of each of the released/emitted gases, thereby enabling detecting and identifying thereof.

According to some embodiments, if no organic outgassing is detected or if the organic outgassing is below a predetermined threshold, the method may optionally include a step 208a to transfer the sample to the main chamber for inspection. According to some embodiments, the instructions including transferring the sample into the main chamber of the tool may be based, at least in part, on one or more of: detecting, by the TOF-RGA sensor, a predefined level of outgassing, detecting a substantially an insignificant level of outgassing, determining, based the analysis of the TOF-RGA sensor, that the outgassing from the sample is substantially devoid of undesired gases, and the like, or any combination thereof. As a non-limiting example, step 208a may optionally include detecting, based at least in part on the TOF-RGA sensor, that the sample has substantially reached a steady state (i.e., the sample has substantially finished outgassing) and/or reached a substantially insignificant outgassing level therefrom.

According to some embodiments, if organic outgassing is detected and/or is above a predetermined threshold, the method may optionally include a step 208b of maintaining the sample in the load-lock chamber for a pre-determined amount of time (time out), such as up to 5 minutes or up to 2 minutes.

During the time out, in step 210 the TOF-RGA sensor continues to perform residual mass analysis. If during or upon completion, organic outgassing is no longer detected the method may optionally include a step 212a of transferring the sample to the main chamber for inspection.

According to some embodiments, if organic outgassing is no longer detected or is below a predetermined threshold level, the method may optionally include a step 212a of transferring the sample to the main chamber for inspection.

Alternatively, if organic outgassing is still detected and/or is above the predetermined threshold, the method may include a step 212b of removing the sample from the load-lock chamber without transferring to the main chamber According to some embodiments, the one or more undesired gases may include, among others, organic outgassing, such as but not limited to hydrocarbons outgassing, and the like, or any combination thereof. Additionally, or alternatively, if organic outgassing is still detected and/or is above the predetermined threshold, the method may include a step 212c of issuing an alert, such as a sound alert or an output message alert.

### EXAMPLES

Reference is made to FIG. 3, which is an experimentally obtained plot 300 of a partial pressure as a function of an atomic mass unit (amu) detected by a TOF-RGA sensor positioned in a vacuum chamber. The tested sample included an organic contaminant resembling photoresist outgassing examined under a pressure of about 2e⁻⁶Torr.

The TOF-RGA sensor utilized had a a mass range of about 1-300 amu. As depicted in plot 300, while a characteristic signature of a particular material released/emitted from the tested sample is not readily seen by detecting the outgassing in the load-lock chamber, the TOF-RGA sensor can identify that the sum of the outgassing has reached a predetermined threshold, such as about 50 amu or higher (schematically marked by “Σ=contamination”). Put differently, in some embodiments, outgassing contamination may be defined as gases, or any other materials, having a sum of a range of peaks at about 50 amu and higher. Gases detected at 50 amu and higher include hydrocarbon-based contaminations. That is by utilizing a TOF-RGA sensor a rapid detection of a superposition of organic/hydrocarbon-based contaminations released/emitted from a tested sample is achieved thus advantageously enabling minimize cross-contamination between different samples and facilitate health of the tool, without requiring prolonged measurement stop during production.

Reference is made to FIG. 4, which is an experimentally obtained plot 400 of a partial pressure as a function of an amu detected by a quadrupole-RGA sensor positioned in a vacuum chamber. The tested sample included an organic contaminant resembling photoresist.

The quadrupole-RGA sensor utilized in the experimental setup had a mass range of about 1-300 amu. As depicted in FIG. 4, the quadrupole-RGA sensor is characterized by high sensitivity of outgassing detection. Put differently, as seen experimentally obtained plot 400 provides characteristic signatures of materials released/emitted from the tested sample. However, the higher sensitivity comes at a price of prolonged equilibration period that interfere with production times and cost, while most often knowing the exact identity of the outgassing is typically not essential

In the description and claims of the application, the words “include” and “have”, and forms thereof, are not limited to members in a list with which the words may be associated.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications, and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

What is claimed is:

1. A system for detecting and/or minimizing outgassing contamination released from a sample into a characterization tool, the system comprising:
a main chamber comprising a characterization tool configured to characterize a sample, wherein operation conditions of the characterization tool comprise reaching a vacuum level of 10⁻⁵ Torr or higher;
a load-lock chamber configured to receive the sample;
a time-of-flight residual gas analyzer (TOF-RGA) sensor in fluid flow communication with the load-lock chamber, wherein the TOF-RGA sensor is configured to provide a real-time measurement of an amount of outgassing released from the tested sample in the load-lock chamber; and
a controller configured to receive measurements from the TOF-RGA sensor, to compute an amount of outgassing detected in the load-lock chamber and to trigger transfer of the sample from the load-lock chamber to the main chamber only if the amount of outgassing detected in the load-lock chamber is at or below a predetermined threshold, thereby reducing a risk for cross-contamination of other/next tested samples and facilitating maintaining tool health and to transfer the sample into the characterization chamber.

2. The system of claim 1, wherein computing the amount of outgassing comprises computing a sum of a predetermined range of peaks.

3. The system of claim 2, wherein the computing comprises distinguishing between water and organic outgassing,
4. The system of claim 1, further comprising a pump configured to generate a vacuum in the load-lock chamber, and a pressure monitor configured to monitor the pressure within the load-lock chamber.

5. The system of claim 4, wherein the controller is configured to trigger measurements by the TOF-RGA sensor, when signals provided by the pressure monitor indicate that a first predetermined pressure level has been obtained.

6. The system of claim 4, wherein the controller is configured to trigger transfer of the sample when signals provided by the pressure monitor indicate that a second predetermined pressure, essentially equal to the pressure in the main chamber, or when signals provided by the pressure monitor indicate that a predetermined third threshold value has been reached; or when signals provided by the pressure monitor indicate that a predetermined amount of time has passed.

7. The system of claim 1, wherein an outgassing sensitivity of the TOF-RGA sensor is in a range of 10⁻⁴ Torr to 10⁻¹⁰ Torr.

8. The system of claim 1, wherein a measurement time of the TOF-RGA sensor to provide the composition of gases is 30 seconds or less.

9. The system of claim 1, wherein a measurement time of the TOF-RGA sensor to provide the composition of gases is 5 seconds or less.

10. The system of claim 1, wherein the TOF-RGA sensor provides the real-time measurement of the composition of gases having a pressure of up to 10⁻⁴ Torr.

11. The system of claim 1, wherein the TOF-RGA sensor is replaceable.

12. The system of claim 1, wherein the characterization tool is selected from:
a scanning electron microscope (SEM), a focused ion beam (FIB), a transmission electron microscope (TEM), a scanning probe microscope (SPM).

13. The system of claim 1, wherein the tested sample comprises a photoresist or an organic residue from a previous process step.

14. The system of claim 1, wherein the controller is further configured to trigger an alert if the amount of outgassing exceeds the predetermined threshold for a predetermined amount of time.

15. A method for detecting and/or minimizing outgassing from a sample, comprising:
positioning a tested sample into a load-lock chamber of a characterization tool;
activating a pump associated with the load-lock chamber to achieve a first pre-determined vacuum level in the load-lock;
upon reaching the first pre-determined vacuum level, performing a residual mass analysis by a TOF-RGA sensor fluidly connected to the load-lock chamber, to identify an amount of gasses released from the tested sample into the load-lock chamber; and
transferring the sample into a main chamber of the characterization tool only if the amount of gasses detected is below a predetermined threshold value, thereby reducing a risk for cross-contamination of other/next tested samples and facilitating maintaining tool health.

16. The method of claim 15, further comprising, if the amount of gasses detected is above the predetermined threshold value, maintaining the tested sample in the load-lock chamber for a predetermined amount of time.

17. The method of claim 16, if during the predetermined amount of time, the amount of gasses detected is at or below the predetermined threshold value, transferring the sample into the main chamber of the characterization tool.

18. The method of claim 16, if during the predetermined amount of time, the amount of gasses detected remains above the predetermined threshold value, removing the sample from the load-lock chamber.

19. The method of claim 15, wherein computing the amount of outgassing comprises computing a sum of a predetermined range of peaks.

20. The method of claim 15, wherein the sample is a wafer.
